SEQUENCE LISTING (7 sequences)

SEQ ID NO: 1 - DNA, 1401 bp, Homo sapiens
atggtctcccaggccctcaggctcctctgccttctgcttgggcttcagggctgcctggctgcaggcggggtcgctaaggcctcaggaggagaaacacgggacatgccgtggaagccggggcctcacagagtcttcgtaacccaggaggaagcccacggcgtcctgcaccggcgccggcgcgccaacgcgttcctggaggagctgcggccgggctccctggagagggagtgcaaggaggagcagtgctccttcgaggaggcccgggagatcttcaaggacgcggagaggacgaagctgttctggatttcttacagtgatggggaccagtgtgcctcaagtccatgccagaatgggggctcctgcaaggaccagctccagtcctatatctgcttctgcctccctgccttcgagggccggaactgtgagacgcacaaggatgaccagctgatctgtgtgaacgagaacggcggctgtgagcagtactgcagtgaccacacgggcaccaagcgctcctgtcggtgccacgaggggtactctctgctggcagacggggtgtcctgcacacccacagttgaatatccatgtggaaaaatacctattctagaaaaaagaaatgccagcaaaccccaaggccgaattgtggggggcaaggtgtgccccaaaggggagtgtccatggcaggtcctgttgttggtgaatggagctcagttgtgtggggggaccctgatcaacaccatctgggtggtctccgcggcccactgtttcgacaaaatcaagaactggaggaacctgatcgcggtgctgggcgagcacgacctcagcgagcacgacggggatgagcagagccggcgggtggcgcaggtcatcatccccagcacgtacgtcccgggcaccaccaaccacgacatcgcgctgctccgcctgcaccagcccgtggtcctcactgaccatgtggtgcccctctgcctgcccgaacggacgttctctgagaggacgctggccttcgtgcgcttctcattggtcagcggctggggccagctgctggaccgtggcgccacggccctggagctcatggtcctcaacgtgccccggctgatgacccaggactgcctgcagcagtcacggaaggtgggagactccccaaatatcacggagtacatgttctgtgccggctactcggatggcagcaaggactcctgcaagggggacagtggaggcccacatgccacccactaccggggcacgtggtacctgacgggcatcgtcagctggggccagggctgcgcaaccgtgggccactttggggtgtacaccagggtctcccagtacatcgagtggctgcaaaagctcatgcgctcagagccacgcccaggagtcctcctgcgagccccatttccctag

SEQ ID NO: 2 - protein, 406 aa, Homo sapiens [sequence omitted]

SEQ ID NO: 3 - protein, 24 aa, Artificial Sequence
  Synthetic Peptide
ANAFLEELRPGSLERECKEEQCSF

SEQ ID NO: 4 - protein, 27 aa, Artificial Sequence
  Synthetic Peptide
IVGGKVCPKGECPWQVLLLVNGAQLCG

SEQ ID NO: 5 - protein, 11 aa, Artificial Sequence
  Synthetic Peptide
LFWISYSDGDQ

SEQ ID NO: 6 - protein, 18 aa, Artificial Sequence
  Synthetic Peptide
GATALELMVLNVPRLMTQ

SEQ ID NO: 7 - protein, 21 aa, Artificial Sequence
  Synthetic Peptide
KVGDSPNITEYMFCAGYSDGS

(19) United States
(12) Patent Application Publication
(10) Pub. No.: US 2011/0162094 A1
(43) Pub. Date: Jun. 30, 2011

(54) MODIFIED HUMAN FACTOR VII/VIIA AND PHARMACEUTICAL COMPOSITION CONTAINING THE SAME

(75) Inventors: Emmanuel Nony, Antony (FR); Sami Chtourou, Elancourt (FR); Nicolas Bihoreau, Orsay (FR)

(73) Assignee: LFB BIOTECHNOLOGIES, Les Ulis (FR)

(21) Appl. No.: 12/664,733

(22) PCT Filed: Jun. 12, 2008
(86) PCT No.: PCT/FR08/51055

§ 371 Date: Jan. 25, 2010

(30) Foreign Application Priority Data
Jun. 15, 2007 (FR) 0755775

(51) Int. Cl. A01K 67/027; C12N 9/64; C07H 21/00; C12N 15/63; C12N 5/10; C12N 1/00; A01K 67/00; A01H 5/00; A01K 67/033; A61K 38/48; A61P 7/04

(52) U.S. Cl. 800/7; 435/226; 536/23.2; 435/320.1; 435/325; 435/243; 800/13; 800/295; 800/14; 424/94.64

(57) ABSTRACT

Modified factors VII/VIIa with a high stability, nucleic acids encoding such modified factors VII/VIIa, and methods of preparing the same.

### FIELD OF THE INVENTION

The field of the present invention relates to the preparation of human factors VII (FVII)/activated factors VII (FVIIa) to be used as active agents for drugs. The present invention relates more particularly to modified factors VII/VIIa with a high stability, to nucleic acids encoding such modified FVII/VIIa and to methods of preparing the same.

### PREVIOUS ART

Factor VII (FVII) is a vitamin K-dependent glycoprotein which under its activated form (FVIIa) takes part to the clotting process by activating factor X and factor IX in the presence of calcium and tissue factor. FVII is secreted in the form of a single peptide chain having 406 amino acid residues, which molecular weight is of about 50 kDa. FVII comprises four distinct structural domains: the N-terminal γ-carboxylic domain (Gla), two epidermal growth factor-like domains (EGF-like), as well as a serine protease domain. The activation of FVII to FVIIa is characterized by the cleavage of the Arg152-Ile153 bond (Arginine 152-Isoleucine 153). FVIIa is therefore composed of a 152 amino acid light chain with a molecular weight of about 20 kDa and of a 254 amino acid heavy chain with a molecular weight of about 30 kDa bound together through a single disulfide bridge (Cysteine 135-Cysteine 262).

FVII/VIIa is used for treating patients that are hemophiliac and suffer from a factor VIII deficiency (haemophilia A) or from a factor IX deficiency (haemophilia B), as well as for patients having other clotting factor deficiencies, for example a heritable FVII deficiency. FVII/VIIa is also recommended for treating cerebrovascular accidents.

The oldest method for obtaining FVIIa concentrates consisted in purifying FVIIa from fractionation-derived plasma proteins.

To this end, EP 0 346 241 describes how to prepare a FVIIa-enriched fraction, resulting from the absorption, then the elution of a plasma protein fractionation by-product containing FVII and FVIIa and other proteins such as factors IX, X and II, including PPSB preeluate (P=prothrombin or FII, P=proconvertin or FVII, S=Stuart factor or FX and B=antihaemophilic B factor or FIX).

Likewise, EP 0 547 932 describes a method for preparing a highly pure FVIIa concentrate substantially free of vitamin K dependent-factors and of FVIII.

One of the major drawbacks of such methods for obtaining FVII/VIIa from blood plasma is that they only enable to obtain small amounts of product. On the other hand, a major drawback is the sensitivity of the obtained products that do systematically provide truncated forms and thus are less active and more likely to cause unwanted side effects. Moreover, the availability of plasma collected from blood donors remains limited.

For this reason, DNA encoding human factor VII was isolated as early as in the 80s (Hagen and al. (1986); Proc. Natl. Acad. Sci. USA; April 83(8):2412-6) and the corresponding protein was expressed into BHK mammal cells (Baby Hamster Kidney) (document EP 0 200 421). The French application FR 06 04872 filed by the applicant also describes the production of FVIIa in a transgenic animal.

These production methods enable to obtain secured proteins against a possible contamination by viruses or other pathogens. Such methods enable to obtain proteins which primary sequence is the same as the human primary sequence.

Commercial preparations of recombinant human FVIIa are currently available under the trade name NovoSeven® (NovoNordisk™). Relatively high doses, as well as frequent intravenous administrations are needed to achieve and maintain the desired therapeutic or prophylactic effect. Therefore, such a treatment still remains both restrictive for the patients and very expensive.

Moreover, it has been shown that FVII/VIIa is a protein that is sensitive to proteolytic cleavage resulting in the formation of a plurality of decomposition products devoid of any clotting activity (atypical cleavages). Atypical cleavages may occur in various steps of the preparation method but also during the storage of FVII/VIIa. Decomposition products have been observed for both the plasma-derived FVII/VIIa and the FVII/VIIa produced using gene recombinant procedures. Atypical cleavages may be involved before FVII be activated to FVIIa, for example during the production and purification of FVII, during the activation step as such or during the purification and/or storage of the activated product (FVIIa).

The European patent EP 0 370 036 relates to a FVII/VIIa that was modified on the lysine, arginine, isoleucine and/or tyrosine residues involved in the FVII/VIIa atypical cleavage to reduce FVII/VIIa atypical cleavages and thus obtain a more stable FVII/VIIa. However this patent does only partially solve the difficulty of obtaining a more stable FVII/VIIa as it does not address the problematic alteration in the FVII/VIIa conformation due to the modification of the amino acids that are involved in the atypical cleavage. This patent neither describes nor suggests the way to obtain a FVII/VIIa that would be modified at the atypical cleavage site level and the conformation of which would not be or would be little affected by the amino acid sequence modification.

Despite the existence of documents about human FVII/VIIas that were modified, especially at the atypical cleavage site level, there is still a crucial need for new human FVII/VIIas having improved properties.

### SUMMARY OF THE INVENTION

The present invention relates to highly stable factors FVII/VIIa, modified on at least two amino acid residues selected from lysine 38, arginine 290 and arginine 315, said amino acid residues being (i) replaced by a distinct amino acid residue or (ii) deleted.

The present invention further relates to nucleic acids encoding the hereabove modified factors FVII/FVIIa, recombinant vectors in which said nucleic acids are inserted, host cells transformed with said nucleic acids or said recombinant vectors and genetically modified organisms expressing said modified factors FVII/VIIa.

The present invention further relates to a method for preparing a modified factor FVII/FVIIa such as defined hereabove.

The present invention also relates to the use of the hereabove modified factors FVII/FVIIa for preparing drugs, as well as pharmaceutical compositions comprising said modified factors FVII/VIIa.

### DESCRIPTION OF THE FIGURES

FIG. 1: MALDI-TOF mass spectrum under native conditions showing the amino acid sequences resulting from the FVII atypical cleavage.

FIG. 2: MALDI-TOF mass spectrum under reducing conditions showing the amino acid sequences resulting from the FVII atypical cleavage.

FIG. 3: Molecular modeling illustrating the structural superposition of native human FVII containing lysine 38 (in white) and of modified human FVII containing glutamine at position 38 (in black) using the Sybyl 7.2 software (Tripos).

FIG. 4: Molecular modeling illustrating the structural superposition of native human FVII containing arginine 290 (in white) and of modified human FVII containing glutamine at position 290 (in black) using the Sybyl 7.2 software (Tripos).

FIG. 5: Molecular modeling illustrating the structural superposition of native human FVII containing arginine 315 (in white) and of modified human FVII containing glutamine at position 315 (in black) using the Sybyl 7.2 software (Tripos).

### DESCRIPTION OF THE INVENTION

The present invention provides new modified factors FVII/VIIa which are highly stable, both (i) during the storage period and (ii) in vivo after their administration to the patients.

Surprisingly, the applicant showed that some mutations of the amino acid residues lysine 38 (Lys 38, K38), arginine 290 (Arg290, R290) and arginine 315 (Arg315, R315) within the amino acid sequence of the natural human FVII/FVIIa do not alter or do little alter the conformation of the thus modified human FVII/VIIa, as compared to a natural human FVII/FVIIa.

In addition, the applicant showed that a modified FVII/VIIa of the invention, the three-dimensional conformation of which is very similar to and sometimes even the same as the three-dimensional conformation of natural human FVII/FVIIa, possesses improved properties, including a reduced atypical cleavage rate, better production yields, a diminished clearance and a higher stability as compared to natural human FVII/VIIa, while retaining a conformation close to that of natural human FVII/VIIa.

As used herein, an “atypical cleavage” means any peptide bond cleavage, except the cleavage of the activation site (cleavage of the Arg₁₅₂-Ile₁₅₃ bond), occurring on the FVII or FVIIa molecule. These atypical cleavages relate especially to the amino acids lysine 38 (lysine-38-leucine-39 bond), arginine 290 (arginine-290-glycine-291 bond) and arginine 315 (arginine-315-lysine-316 bond) and do cause structural modifications leading to an alteration of the FVII/VIIa pharmacokinetic properties.

As used herein, a “production yield” means the amount of structurally conformable and active FVII/VIIa produced per volume of fermenter (or bioreactor) or per volume of milk from transgenic animals or per weight of any biomass (animal, vegetable, bacterial or insect cells). The production cost for a thus mutated FVII/VIIa is therefore significantly lower than that of a FVII/VIIa the primary sequence of which is the same as the native human FVII/VIIa sequence.

As used herein, the “clearance” means the fraction of a fully purified theoretical volume, that is to say that does not contain FVII/VIIa anymore per unit of time. The FVII/FVIIa clearance represents a plasma purification coefficient. This corresponds to the ability of an organ to totally remove FVII/FVIIa from a given volume of arterial plasma per unit of time. FVII/FVIIa clearance is the apparent volume (virtual volume) of arterial plasma fully cleared from FVII/FVIIa given per unit of time.

As used herein, the “stability” means the ability for FVII/VIIa to retain its chemical, physical, structural, conformational and/or biopharmaceutical properties for all its shelf life.

As used herein, the “conformation” means the tertiary structure of a protein, that is to say the folding in the space of the polypeptide chain. It is frequently referred to as a three-dimensional structure, or a 3D structure. The conformation of a protein is intimately associated with its biological activity, which explains that when its structure is altered, the protein loses its biological activity and becomes denatured. As used herein, an “alteration in the conformation” therefore means any modification relative to the three-dimensional structure of a protein which leads to a loss of the biological activity of said protein.

The biological activity of the FVII/VIIa of the present invention may be quantified by measuring the ability for FVII/VIIa to induce blood clotting by means of a FVII-deficient plasma and thromboplastin, as for example described in the U.S. Pat. No. 5,997,864. In the assay described in the U.S. Pat. No. 5,997,864, the biological activity is expressed by a decrease in the clotting time as compared to the control sample, and is converted into “FVII/VIIa units” by comparison with a human serum standard containing 1 unit/ml of FVII/VIIa activity.

The FVII/VIIa of the invention has posttranslational modification characteristics similar to that of the native human FVII/VIIa, but may also have posttranslational modifications that differ from that of the plasma-derived native human FVII/VIIa so as to improve its chemical, physical, structural, conformational and/or biopharmaceutical properties.

In its broadest aspect, the present invention relates to provide a human FVII/VIIa modified as compared to the peptide sequence of the native human FVII/VIIa having at least two amino acids selected from lysine 38, arginine 290 and arginine 315 which are substituted or deleted, wherein:
(i) lysine 38 is replaced by an amino acid selected from glutamine, alanine, glutamic acid, glycine, isoleucine, leucine, methionine, histidine, phenylalanine, proline, serine, threonine, tryptophan, tyrosine or valine, preferably glutamine, histidine or glutamic acid;
(ii) arginine 290 is replaced by an amino acid selected from glutamine, alanine, glutamic acid, asparagine, glycine, isoleucine, leucine, methionine, histidine, phenylalanine, proline, serine, threonine, tryptophan, tyrosine or valine, preferably glutamine, histidine, asparagine or glutamic acid, and/or
(iii) arginine 315 is replaced by an amino acid selected from glutamine, alanine, glutamic acid, asparagine, glycine, isoleucine, leucine, methionine, histidine, phenylalanine, proline, serine, threonine, tryptophan, tyrosine or valine, preferably glutamine, histidine, asparagine or glutamic acid.

In a preferred embodiment of the present invention, FVII/VIIa comprises at least two substitutions selected from lysine 38 replaced by glutamine, arginine 290 replaced by glutamine and arginine 315 replaced by glutamine.

In a first particular embodiment of the present invention, FVII/VIIa comprises a mutation on lysine 38 and arginine 290.

In a second particular embodiment of the present invention, FVII/VIIa comprises a mutation on lysine 38 and arginine 315.

In a third particular embodiment of the present invention, FVII/VIIa comprises a mutation on arginine 290 and arginine 315.

In a fourth particular embodiment of the present invention, FVII/VIIa comprises a mutation on lysine 38, arginine 290 and arginine 315.

In a particular embodiment of the present invention, lysine 38 is replaced by glutamine, arginine 290 is replaced by glutamine and arginine 315 is replaced by glutamine.

The FVII/VIIa of the invention may be produced by implementing recombinant DNA technologies (genetic recombination). In general, the nucleic sequence of a nucleic acid (DNA or RNA) encoding a native human FVII/VIIa is modified to encode the desired protein, especially a modified FVII/FVIIa according to the invention. The thus modified nucleic acid may then be inserted into an expression vector, which is then used to transform or transfect a host cell. A nucleic acid encoding a native human FVII/VIIa is illustrated by the nucleic acid of SEQ ID NO 1.

Hence, the present invention also relates to provide a nucleic acid encoding a modified human FVII/VIIa according to the present invention, as well as a nucleic acid of a complementary sequence. A nucleic acid encoding a modified human FVII/VIIa according to the present invention may be produced or synthesized using any of the known traditional techniques that do belong to the general knowledge of the man skilled in the art. As an illustration, a nucleic acid encoding a modified human FVII/VIIa according to the present invention may be obtained by a genetic recombination from the nucleic acid encoding native human FVII/VIIa. Preferably, the nucleic acid encoding the modified human FVII/VIIa is obtained by site specific mutagenesis from the nucleic acid encoding native human FVII/VIIa. Site specific mutagenesis techniques are well known from the man skilled in the art and enable to obtain a DNA encoding the desired modified human FVII/VIIa. Site specific mutagenesis techniques may be implemented for instance, that are identical to or derived from the site specific mutagenesis technique described by Michael Smith in 1978 (Smith and al.; “Mutagenesis at a specific position in a DNA sequence”; J Biol. Chem. (1978) Sep. 25; 253(18):6551-60). Advantageously, the FVII/VIIa of the invention is a polypeptide having at least two amino acid residues, selected from amino acids lysine 38, arginine 290 and arginine 315 of the native human FVII of SEQ ID NO 2, which are replaced by amino acids selected to this end, or are deleted.

In a particular embodiment, a modified FVII/VIIa according to the present invention may be obtained from a variant of native human FVII/VIIa, provided that this variant is not more immunogenic than native human FVII/VIIa. Thus, the peptide sequence of this variant may present at least 70% amino acid identity, and advantageously at least 80% or 90%, and even more advantageously at least 99% amino acid identity to the peptide sequence of native human FVII and comprises at least two amino acid residues selected from amino acids lysine 38, arginine 290 and arginine 315, according to the amino acid numbering of the native human FVII of SEQ ID NO 2, which are mutated with amino acids selected to this end, or are deleted. Such a variant has substantially a similar or a better biological activity as compared to native human FVII/VIIa.

For the purpose of the present description, a “nucleotide sequence” may be used for meaning either a polynucleotide or a nucleic acid. A “nucleotide sequence” includes the genetic material as such and therefore is not limited to the information about the sequence.

As used herein, a “nucleic acid”, a “polynucleotide”, an “oligonucleotide” or a “nucleotide sequence” include RNA, DNA, cDNA sequences or RNA/DNA hybrid sequences of more than one nucleotide, either in the single-stranded form or in the double-stranded form. A “nucleotide” means natural nucleotides [Adenine (A), Thymine (T), Guanine (G), Cytosine (C) and Uracil (U)].

For the purpose of the present invention, a first polynucleotide is considered as being “complementary” to a second polynucleotide when each base of the first nucleotide is paired to the complementary base of the second polynucleotide which has a reverse orientation. Complementary “bases” are A with T (or A with U), and C with G.

According to the present invention, a first nucleic acid having at least 90% identity to a second reference nucleic acid, will have at least 90%, preferably at least 91%, 92%, 93%, 94%, 95%, 96%, 97%, 97.5%, 98%, 98.3% 98.6%, 99%, 99.6% nucleotide identity to said second reference nucleic acid. According to the present invention, a first polypeptide having at least 90% identity to a second reference polypeptide, will have at least 90%, preferably at least 91%, 92%, 93%, 94%, 95%, 96%, 97%, 97.5%, 98%, 98.3% 98.6%, 99%, 99.6% amino acid identity to said second reference polypeptide.

The “identity percentage” between two nucleic acid sequences, or between two amino acid sequences, as defined in the present invention, is determined by comparing the two optimally aligned sequences through a comparison window.

The portion of the nucleotide sequence or amino acid sequence within the comparison window may thus comprise additions or deletions (for example gaps) as compared to the reference sequence (which does not comprise these additions or deletions) so as to obtain an optimal alignment between the two sequences.

The identity percentage is calculated by determining the number of positions at which an identical nucleic base, or an identical amino acid is observed for the two compared sequences, then by dividing the number of positions where there is an identity between the two nucleic bases, or between the two amino acids, by the total number of positions within the comparison window, lastly by multiplying the result by hundred to obtain the identity percentage of nucleotides, or of amino acids between both sequences.

An optimal sequence alignment for comparison may be calculated by computer programs using known algorithms.

Most preferably, said sequence identity percentage is determined using the CLUSTAL W software (version 1.82) which parameters are set as follows: (1) CPU MODE=ClustalW mp; (2) ALIGNMENT=“full”; (3) OUTPUT FORMAT=“aln w/numbers”; (4) OUTPUT ORDER=“aligned”; (5) COLOR ALIGNMENT=“no”; (6) KTUP (word size)=“default”; (7) WINDOW LENGTH=“default”; (8) SCORE TYPE=“percent”; (9) TOPDIAG=“default”; (10) PAIRGAP=“default”; (11) PHYLOGENETIC TREE/TREE TYPE=“none”; (12) MATRIX=“default”; (13) GAP OPEN=“default”; (14) END GAPS=“default”; (15) GAP EXTENSION=“default”; (16) GAP DISTANCES=“default”; (17) TREE TYPE=“cladogram” and (18) TREE GRAP DISTANCES=“hide”.

The present invention also relates to provide an expression vector wherein a nucleic acid was inserted, encoding a modified human FVII/VIIa according to the present invention.

The expression vector used in the present invention may comprise a promoter capable of directing the transcription of the nucleic acid encoding the FVII/VIIa of the invention. Promoters that are traditionally used for mammal cell cultures comprise viral promoters and cell promoters that are well known from the state of the art. The expression vector may further comprise splicing sites located downstream the promoter and upstream the insertion site of the DNA sequence encoding a FVII/VIIa of the invention. The expression vector may further comprise a polyadenylation sequence located downstream the insertion site of the DNA sequence encoding the FVII/VIIa of the invention. The expression vector may further comprise any type of DNA sequence useful for the expression, selection and/or insertion of the FVII/VIIa, of the DNA sequence encoding the FVII/VIIa of the invention and/or of the expression vector containing the DNA sequence encoding the FVII/VIIa of the invention.

The present invention also relates to provide a cell that is transformed to produce a modified human FVII/VIIa of the present invention. The transformed cells are obtained by transferring a nucleic acid encoding a modified human FVII/VIIa of the present invention in the genome of a host cell, preferably so as to express this DNA sequence by the thus transformed cell. Suitable cell transformation methods are well known from the man skilled in the art. These methods comprise, without being limited thereto, the use of liposomes, the use of polyethylene glycol (PEG), the use of DEAE-dextran, the use of calcium phosphate, the use of viruses (mostly retroviruses), the use of a DNA gun, cell fusion, microinjection, electroporation, etc.

The present invention therefore also relates to a cell transformed with a nucleic acid encoding a human FVII/VIIa modified as defined hereabove and expressing said modified human factor VII/VIIa. Preferably, said transformed cell is a mammal transformed cell, and especially a murine, a bovine, a caprine, a porcine, a non human primate transformed cell or a human transformed cell.

A modified human FVII/VIIa according to the present invention may be obtained from a cell transformed as per the present invention and cultivated. As an example, following cells may be mentioned: BHK (Baby Hamster Kidney) and especially BHK tk⁻ts13 (CRL 10314, Waechter and Baserga, Proc. Natl. Acad. Sci. USA 79:1106-1110, 1982), CHO (ATCC CCL 61), COS-1 (ATCC CRL 1650), HEK293 (ATCC CRL 1573; Graham and al., J. Gen. Virol. 36:59-72, 1977), Rat Hep I (Rat hepatoma; ATCC CRL 1600), Rat Hep II (Rat hepatoma; ATCC CRL 1548), TCMK (ATCC CCL 139), Human lung (ATCC HB 8065), NCTC 1469 (ATCC CCL 9.1) and DUKX cells (CHO cell line) (Urlaub and Chasin, Proc. Natl. Acad. Sci. USA 77:4216-4220, 1980), cells YB2/0, cells 3T3, cells Namalwa, or BHK cells adapted for a serum-free culture (U.S. Pat. No. 6,903,069).

The present invention also relates to a genetically modified organism to produce a modified human factor VII/VIIa of the present invention. According to the definition given by the European Union, a “genetically modified organism” is an organism (except human being) the genetic material of which has been modified in a way that could not occur naturally by multiplication and/or recombination. In the context of the present invention, a genetically modified organism does integrate the DNA sequence encoding a FVII/VIIa of the invention and does express said DNA sequence of the modified human FVII so as to produce said modified human FVII/VIIa of the invention. The genetically modified organism is a microorganism, an animal or a plant. The present invention therefore further relates to a genetically modified organism comprising in its genome a nucleic acid encoding a human factor VII/VIIa modified such as defined in the present description, and expressing said modified human factor VII/VIIa.

A microorganism is a microscopic organism, and may be either a bacterium, a yeast or a virus. The bacterium may be, for example, Bacillus subtilis (Palva and al. (1982) Proc. Natl. Acad. Sci. USA 79:5582; EP 0 036 259 and EP 0 063 953; WO 84/04541); Escherichia coli (Shimatake and al. (1981) Nature 292:128; Amann and al. (1985) Gene 40:183; Studier and al. (1986) J. Mol. Biol. 189:113; EP 0 036 776, EP 0 136 829 and EP 0 136 907); Streptococcus cremoris (Powell and al. (1988) Appl. Environ. Microbiol. 54:655]; Streptococcus lividans [Powell and al. (1988) Appl. Environ. Microbiol. 54:655); Streptomyces lividans (U.S. Pat. No. 4,745,056). The yeast may be, for example, Candida (Kurtz and al. (1986) Mol. Cell. Biol. 6:142; Kunze and al. (1985) J. Basic Microbiol. 25:141); Hansenula (Gleeson and al. (1986) J. Gen. Microbiol. 132:3459; Roggenkamp and al. (1986) Mol. Gen. Genet. 202:302); Kluyveromyces (Das and al. (1984) J. Bacteriol. 158:1165; De Louvencourt and al. (1983) J. Bacterial. 154:1165; Van den Berg and al. (1990) Bio/Technology 8:135); Pichia (Cregg and al. (1985) Mol. Cell. Biol. 5:3376; Kunze and al. (1985) J. Basic Microbiol. 25:141; U.S. Pat. Nos. 4,837,148 and 4,929,555); Saccharomyces (Hinnen and al. (1978) Proc. Natl. Acad. Sci. USA 75; 1929; Ito and al. (1983) J. Bacteriol. 153:163); Schizosaccharomyces (Beach and Nurse (1981) Nature 300:706); Yarrowia (Davidow and al. (1985) Curr. Genet. 10:39; Gaillardin and al. (1985) Curr. Genet. 10:49). The virus used may be, for example, a retrovirus such as Avian Leukosis Virus, Bovine Leukemia Virus, Murine Leukemia Virus, Mink-Cell Focus-Inducing Virus, Murine Sarcoma Virus, Reticuloendotheliosis Virus and Rous Sarcoma Virus.

An animal as defined in the present invention is a non human, living pluricellular organism, of the eukaryotic type, without any chloroplast. In a preferred embodiment, the genetically modified organism used in the present invention is a mammal, preferably a female rabbit. Advantageously, the modified human FVII/VIIa of the present invention may be produced in the mammary glands of a mammal, preferably a female rabbit, under the control of a specific promoter enabling the expression of said FVII/VIIa in the milk of said female rabbit.

A method for producing a recombinant or a transgenic FVII/VIIa in the milk of a transgenic animal may comprise the following steps: a DNA molecule comprising a gene encoding the modified human FVII/VIIa of the invention, said gene being under the control of a promoter of a protein that is naturally secreted in the milk (such as the casein gene promoter, the beta-casein gene promoter, the lactalbumin gene promoter, the beta-lactoglobulin gene promoter or the WAP gene promoter) is integrated into an embryo of a non human mammal. The embryo is then placed in a female mammal of the same species. Once the mammal derived from the embryo has sufficiently developed, the mammal lactation is elicited and the milk is collected. The milk then contains said recombinant or transgenic FVII/VIIa.

An example of a protein preparation method in the milk of a female mammal other than a human being is given in EP 0 527 063, the teaching of which may be taken into account to produce the protein of the invention. A plasmid comprising the WAP gene promoter (Whey Acidic Protein) is obtained by introducing a WAP gene promoter-containing sequence, this plasmid being prepared so as to be able to receive a foreign gene placed under the control of the WAP gene promoter. The plasmid comprising said promoter and the gene encoding the protein of the invention are used to produce transgenic female rabbits, by microinjecting into the male pronucleus female rabbit embryos. The embryos are then transferred into the oviduct of hormonally prepared females. The presence of the transgenes is revealed by Southern blot from DNA extracted from the thus obtained transgenic young rabbits. Animal milk concentrations are evaluated using specific radioimmunoassays.

Other documents describe methods for preparing proteins in the milk of a female mammal other than a woman. There are to be mentioned, without being limited thereto, U.S. Pat. No. 7,045,676 (transgenic mouse) and EP 1 739 170 (production in a transgenic mammal of the von Willebrand factor) may be mentioned. These preparation methods do apply to the present invention using DNA from the modified FVII/VIIa of the present invention.

In a particular embodiment, the genetically modified organism is an insect, for example a mosquito, a fly, etc.

As used herein, a “recombinant or transgenic FVII/VIIa” means any FVII/VIIa obtained from a transformed cell or from a genetically modified organism, that is to say from a microorganism, an animal or a plant. By contrast, the FVII/VIIa of the invention is not a plasma-derived FVII/VIIa, that is to say it is not a product purified from a human or animal plasma.

Thus, the FVII/VIIa of the invention is derived from the transcription, then from the translation of a DNA molecule encoding a modified FVII modified of the present invention and produced by a transgenic cell, microorganism, animal or plant. Thus, the recombinant or transgenic FVII/VIIa of the present invention may be obtained using a traditional method well known from the man skilled in the art, enabling the expression of a protein in a biological system.

The present invention also relates to a method for preparing a modified human FVII/VIIa according to the present invention comprising the following steps of:
a) transforming a cell with a nucleic acid encoding a human FVII/VIIa modified such as defined in the present description,
b) culturing the cell obtained in step a) so that said cell does express said factor VII/VIIa, and
c) purifying the modified human factor VII/VIIa expressed by the transformed cell cultured in step b).

The transformed cell is cultured in a suitable medium enabling the expression of FVII/VIIa. Culture media used are selected on purpose by the man skilled in the art depending on the cultured cells. Media that are suitable for cell culture do include IMDM (Iscove's Modified Dulbecco's Medium), DMEM (Dulbecco's Modified Eagle Medium), RPMI 1640 or equivalent. These culture media are mainly composed of inorganic salts, amino acids, vitamins and other components, including glucose for its energy supply and HEPES for its buffering effect, basic complements de base such as amino acids in particular, minerals, trace elements, growth- and metabolic activity-specific molecular complements for each cultured cell type, etc.

The present invention also relates to a method for preparing a modified human FVII/VIIa of the present invention in the milk of a transgenic mammal, comprising the following steps of:
a) providing a transgenic mammal which does express in its mammary glands a nucleic acid encoding a modified human factor VII/VIIa according to the present invention,
b) collecting the milk of the transgenic mammal which contains factor VII/VIIa,
c) purifying the modified human factor VII/VIIa from said collected milk.

Advantageously, the transgenic mammal may be a mouse, a female rat, a female rabbit or a goat. Preferably, the transgenic mammal is a female rabbit.

To provide a transgenic mammal traditional methods may be used, consisting for example in microinjecting a mammal embryo with a DNA sequence encoding a modified human FVII/VIIa of the present invention, introducing said microinjected embryo into the oviduct lumen of a female mammal of the same species, waiting for the birth of the young mammals derived from the microinjected embryo, checking that the transgenic animal does indeed express the modified human FVII/VIIa in its milk.

The FVII/VIIa of the invention may be purified by purification methods well known from the man skilled in the art, including, without being limited thereto, chromatography (ion exchange, affinity, hydrophobic or size exclusion chromatography), electrophoresis-based methods such as preparative isoelectric focusing (IEF), solubility difference (ammonium sulfate precipitation) or extraction (Protein Purification J.-C. Janson and Lars Ryden, editors, VCH Publishers, New York (1989)). Preferably, the FVII/VIIa of the invention may be purified by affinity chromatography on an anti-FVII antibody column or on an anti-FVII aptamer column. An additional purification may be conducted using traditional chemical purification methods, such as HPLC (High performance liquid chromatography). Other purification methods, including barium citrate precipitation, are well known from the man skilled in the art and may be used for purifying the FVII/VIIa of the invention.

As used herein, an “antibody” means an immunoglobulin or an immunologically active fraction thereof, for example the antigen-binding region. An antibody does thus refer to a protein comprising at least one, and preferably two, heavy chain(s) and at least one, preferably two light chain(s).

As used herein, an “aptamer” means a nucleic acid molecule (DNA or RNA) having a tertiary structure enabling it to specifically bind to a protein (Osborne, and al. (1997) Curr. Opin. Chem. Biol. 1: 5-9; and Patel, D. J. (1997) Curr Opin Chem Biol 1:32-46).

It is a further object of the present invention to provide a composition comprising a modified human FVII/VIIa of the present invention.

It is a further object of the present invention to provide a pharmaceutical composition comprising a modified human FVII/VIIa of the present invention and an excipient and/or a pharmaceutically acceptable carrier.

The pharmaceutical composition of the invention may be used for a parenteral, a topical or local administration, and for prophylactic and/or therapeutic applications. Thus, a modified human FVII/VIIa of the present invention is prepared in a form adapted to the chosen administration route, for example in a liquid form or in a freeze-dried form. The pharmaceutical compositions comprising the modified human FVII/VIIa of the present invention may comprise an excipient and/or a pharmaceutically acceptable carrier, preferably aqueous. Many pharmaceutically acceptable excipients and/or carriers may be used, as for example, water, buffered water, a saline solution, a glycine solution and derivatives thereof, as well as agents required for reproducing the physiologic conditions, such as for example buffers and pH-adjusting agents, surfactants such as sodium acetate, sodium lactate, sodium chloride, potassium chloride, calcium chloride, this list being not limitative. In addition, the pharmaceutical composition may be sterilised by sterilisation methods that are well known from the man skilled in the art. Generally speaking, to prepare a pharmaceutical composition according to the invention, the man skilled in the art will advantageously refer to the most recent edition of the European Pharmacopoeia, for example to the 5th Edition of the European Pharmacopoeia, published January 2005, or to the 6th Edition of European Pharmacopoeia, open to the public June 2007.

The modified human FVII/VIIa of the present invention and pharmaceutical compositions comprising the same are particularly useful for preparing drugs. The modified human FVII/VIIa of the present invention and pharmaceutical compositions comprising the same are useful for preparing a drug for treating clotting disorders in a patient. Clotting disorders to be treated with a pharmaceutical composition of the invention comprise, without being limited thereto, multiple hemorrhagic trauma, as for example haemophilia A and B or bleedings caused by anticoagulant overdosage.

The modified Human FVII/VIIa according to the present invention may be used alone or in combination with one or more other pharmaceutically active molecule(s).

### EXAMPLES

### Example 1

### Human FVII Three-Dimensional Model

The human FVII three-dimensional model was conceived based on an exhaustive study of all crystallized structures available within the Protein Data Bank (PDB). 27 FVII structures were analyzed according to various parameters such as the expression system, the heavy and light chain integrity, the tissue factor occurrence, the resolution, the O- and N-glycosylation occurrences, the γ-carboxylation occurrence and the publication date to the Protein Data Bank (PDB). Based on this study, the protein structure was constructed after corrections, assembling and minimization of the structures. The software suite used was Sybyl v7.2 (Tripos, Inc.). Sybyl is a modeling software that relies on the total energy minimization, so as to thus define the most stable structure and so the most plausible. The global minimization step including fixing the protein backbone by simulating the tissue factor occurrence, was performed under following conditions:
stop parameter: energy gradient <0.5 kCal/mol or maximum number of iterations reached=10000
minimization method: Powell
force field: Amber7FF99
method for calculating the glycoprotein charges: Amber7FF99,
method for calculating the ion and active site inhibitor charges: Gasteiger-Hückel
non-bonded cut-off: 8 Å.

### Example 2

### Extraction and Purification of FVII Obtained in the Milk of a Transgenic Female Rabbit

a) FVII Extraction

A volume of 500 ml of non skimmed raw milk was diluted with 9 volumes of sodium phosphate buffer 0.25 M, pH 8.2. After 30 minutes stiffing at room temperature, the FVII-enriched aqueous phase was centrifuged at 10 000 g for 1 hour at 15° C. (Sorvall Evolution RC centrifuge—6700 rpm—rotor SLC-6000). 6 pots of about 835 ml were needed.

After centrifugation, three phases were formed: a lipidic phase on the surface (cream), a clear, FVII-enriched and non lipidic aqueous phase (main phase) and a white solid pellet phase (insoluble casein and calcium compound precipitates).

The FVII-enriched non lipidic aqueous phase was collected with a peristaltic pump to the cream phase. The cream phase was collected aside. The solid phase (precipitate) was removed.

The non lipidic aqueous phase, however still containing very small amounts of lipids, was filtered via a filter sequence (Pall SLK7002U010ZP—glass fiber prefilter with a pore size of 1 μm—followed by Pall SLK7002NXP—Nylon 66 with a pore size of 0.45 μm). At the end of the filtration, the lipidic phase was passed through this filtration sequence which totally retained the lipidic globules of milk, and the filtrate was clear.

The filtered non lipidic aqueous phase was then dialyzed through an ultrafiltration membrane (Millipore Biomax 50 kDa—0.1 m²) to make it compatible with the chromatographic phase. FVII with a molecular weight of about 50 kDa did not filter through the membrane, in opposition to salts, sugars and peptides of milk. In a first step, the solution (approx. 5 000 ml) was concentrated to 500 ml, then an ultrafiltration dialysis, which maintains the volume to a constant level, enabled to remove the electrolytes and to prepare the biological material for the chromatography step. The dialysis buffer was a sodium phosphate buffer 0.025M, pH 8.2.

This non lipidic aqueous phase which comprises FVII can be compared to FVII-tg-enriched lactoserum. This preparation was stored at −30° C. prior to continuing the process.

The non lipidic aqueous phase comprising FVII at the end of this step was perfectly clear and was compatible with the following chromatographic steps.

Approx. 93 000 IU FVII-tg were extracted at this stage. The FVII purity of such preparation was of about 0.2%.

b) FVII Purification

1. Chromatography on Hydroxyapatite Gel

An Amicon 90 column (9 cm diameter—64 cm² section) was filled with BioRad Ceramic Hydroxyapatite type I gel (CHT-I).

The gel was equilibrated in buffer A composed of a mixture of sodium phosphate 0.025 M and sodium chloride 0.04 M, pH 8.0. The totality of the preparation stored at −30° C. was thawed in a water bath at 37° C. until the ice block was completely dissolved, then was injected on the said gel (linear flow rate 100 cm/h, i.e. 105 ml/min). The non-retained fraction was removed by a buffer composed of sodium phosphate 0.025 M and sodium chloride 0.04 M, pH 8.2, until return to baseline (RBL).

Elution of the FVII-containing fraction was effected with buffer B composed of sodium phosphate 0.25 M and sodium chloride 0.4 M, pH 8.0. The eluted fraction was collected until return to baseline.

This chromatography enabled to recover more than 90% of FVII, while removing more than 95% of the lactic proteins. The specific activity (SA) was multiplied by 25. About 85 000 IU FVII with a purity of 4% were available at this stage.

2. Tangential Filtration (100 kDa) and Concentration/Dialysis (50 kDa)

The whole eluate of the previous step was filtered in tangential mode on a 100 kDa ultrafiltration membrane (Pall OMEGA SC 100K—0.1 m²). FVII was filtered through the 100 kDa membrane, while the proteins with a molecular weight higher than 100 kDa could not be filtered.

The filtered fraction was then concentrated to about 500 ml, then dialyzed on the 50 kDA ultrafilter already described in Example 1. The dialysis buffer was sodium chloride 0.15 M.

At this stage of the process, the product was stored at −30° C. before running an ion exchange chromatography.

This step enabled to reduce the charge in proteins having a molecular weight higher than 100 kDa and especially proenzymes. The treatment on the 100 kDa membrane resulted in a retention of about 50% of the proteins, including the high-molecular weight proteins, while filtering 95% of FVII, i.e. 82 000 IU FVII.

This treatment made it possible to reduce the proteolytic hydrolysis risks during the downstream steps.

3. Chromatography on Q-Sepharose® FF Gel

These three successive chromatographies on a Q-Sepharose® Fast Flow (QSFF) ion-exchange gel were conducted to purify the active agent, to enable the activation of FVII to activated FVII (FVIIa) and lastly to concentrate and formulate the FVII composition.

3.1 Q-Sepharose® FF First Step—“High Calcium” Elution

A 2.6 cm diameter column (5.3 cm² section) was filled with 100 ml of Q-Sepharose® FF gel (GE Healthcare).

The gel was equilibrated in 0.05 M Tris buffer, pH 7.5.

The whole fraction stored at −30° C. was thawed in a water bath at 37° C. until the ice block was completely dissolved. The fraction was diluted to a ½ concentration [v/v] with the balance buffer before being injected on gel (flow rate 13 ml/min, with a linear flow rate of 150 cm/h), then the non-retained fraction was removed by running the buffer until return to baseline.

A first protein fraction with a FVII low content was eluted at 9 ml/min (i.e. 100 cm/h) with a Tris 0.05 M and sodium chloride 0.15 M buffer, pH 7.5, and was then removed.

A second protein fraction with a FVII high content was eluted at 9 ml/min (i.e. 100 cm/h) with a Tris 0.05 M, sodium chloride 0.15 M and calcium chloride 0.05 M buffer, pH 7.5.

This second fraction was dialyzed on the 50 kDA ultrafilter already described in Example 1. The dialysis buffer was sodium chloride 0.15 M. This fraction was stored at +4° C. overnight prior to running the column for the second anion exchange chromatography. This step enabled to recover 73% of FVII (i.e. 60000 IU FVII), while removing 80% of the accompanying proteins. It made it also possible to activate FVII to FVIIa.

3.2 Q-Sepharose® FF 2d Step—“Low Calcium” Elution

A 2.5 cm diameter column (4.9 cm² section) was filled with 30 ml of Q-Sepharose® FF gel (GE Healthcare).

The gel was equilibrated in 0.05 M Tris buffer, pH 7.5.

The previous eluted fraction (second fraction), stored at +4° C., was diluted before being injected on gel (flow rate 9 ml/min, with a linear flow rate of 100 cm/h).

A fraction containing FVII of very high purity was eluted at 4.5 ml/min (i.e. 50 cm/h) in Tris 0.05 M, sodium chloride 0.05 M and calcium chloride 0.005 M buffer, pH 7.5.

About 23 000 IU FVII were purified, i.e. 12 mg of FVII.

This step enabled to remove more than 95% of the accompanying proteins (female rabbit's milk proteins).

This eluate, with a purity of more than 90%, had structural and functional characteristics close to that of the native human FVII. II was concentrated and formulated by running through the ion exchange chromatography column for the third time.

3.3 Q-Sepharose® FF 3rd Step—“Sodium” Elution

A 2.5 cm diameter column (4.9 cm² section) was filled with 10 ml of Q-Sepharose® FF gel (GE Healthcare).

The gel was equilibrated in 0.05 M Tris buffer, pH 7.5.

The purified eluted fraction of the previous step was diluted×5 with purified water for injection (WFI) before being injected on gel (flow rate 4.5 ml/min, with a linear flow rate of 50 cm/h).

FVII was then eluted at a flow rate of 3 ml/min (i.e. 36 cm/h) with Tris 0.02 M and sodium chloride 0.28 M buffer, pH 7.0.

A FVII composition as a concentrate was prepared with a purity of more than 95%. The product was compatible with an intravenous injection. The method had a cumulative yield of 22%, which made it possible to purify at least 20 mg of FVII per litre of milk used.

FVII may then be submitted to various structural analyses, such as developed in the following examples.

### Example 3

### Identification of the FVII Atypical Cleavages by MALDI-TOFMS

The mass spectrometry MALDI-TOF MS (Matrix-Assisted Laser Desorption/Ionisation Time of Flight Mass Spectrometry) is a technique enabling to measure the molecular weight of molecules with a high precision.

The tested proteins were mixed to a matrix absorbing at the wavelength of the laser used. The main matrices included α-cyano-4-hydroxycinnamic acid (HCCA) for analyzing peptides, sinapinic acid (SA) for analyzing proteins and 2,5-dihydroxybenzoic acid (DHB) for analyzing oligosaccharides.

The method consisted in irradiating matrix/analyte co-crystals with a laser, which caused the mutual desorption of the matrix molecules and of the analyte molecules. After gas ionization, the analyte molecules reached a time-of-flight detector. As the weight and the time of flight are intimately associated, measuring of the latter enabled to determine the analyte weight. Identifying each protein or each peptide could be done by measuring its weight, as observed in mass spectrometry, and by comparing the theoretical weight derived from the FVII sequence. The apparatus used was a Bruker Autoflex II running in both TOF and TOF/TOF modes.

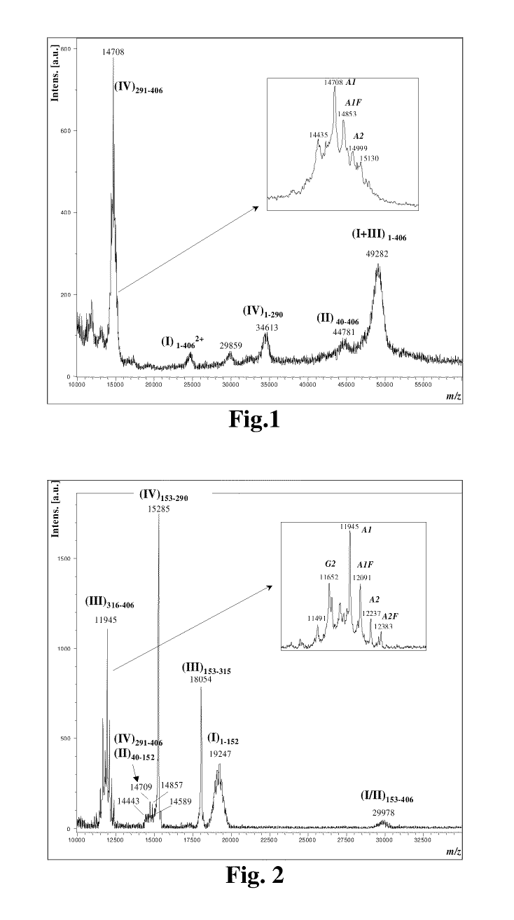

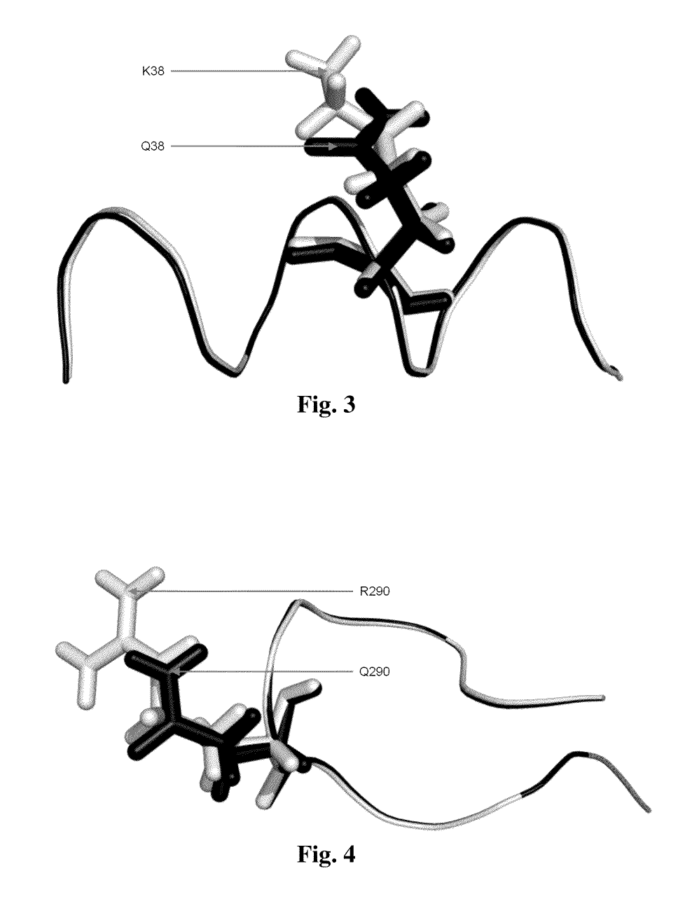

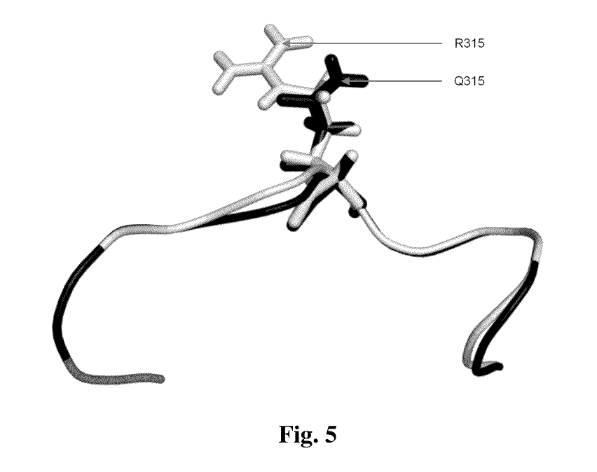

The FVII MALDI-TOF spectrum showed a form at 14.7 kDa (FIG. 1, polypeptide IV) which corresponded to the C-terminal peptide [Gly₂₉₁-Pro₄₀₆] of the heavy chain (HC) containing the Asn₃₂₂ mostly glycosylated by an oligosaccharide of the biantennary monosialylated type (A1) and other glycans (A1F, A2, . . . ). In the spectrum, the presence of the N terminal, complementary form of FVII (FIG. 1, polypeptide IV), ending with an arginine 290, was also observed at 34.6 kDa. Another atypical cleavage was observed at 44.8 kDa (FIG. 1, polypeptide II) which corresponded to the light chain (LC) cleaved after lysine 38, that is to say to a Gla domain-deleted FVII form, which affinity to the tissue factor was thus reduced.

Under reducing conditions (FIG. 2), the presence of FVIIa heavy and light chains, were noted at 29.9 and 19.3 kDa (polypeptide I), respectively. Another 11.9 kDa form was observed corresponding to peptide [Lys₃₁₆-Pro₄₀₆] containing glycosylated Asn₃₂₂. In the native state, this peptide was bound to the N-terminal portion of the protein through a disulfide bridge (Cys₃₁₀-Cys₃₂₉).

All the tested FVII samples did have one or more of these truncated forms. The whole identified forms resulted from serine protease-type cleavages. These various cleavages may thus be of autocatalytic origin.

### Example 4

### Atypical Cleavage Quantification Using Edman Sequencing

FVII N-terminal sequencing was performed on a microsequencer (Procise 491 HT; Applied Biosystem) based on the Edman chemical degradation principle which consists in three steps: coupling—cleavage and conversion, then separation of the amino acids formed on a reversed-phase column. The thus generated N-terminal amino acids are then examined and identified using standard amino acids and compared to the theoretical sequence of the considered protein. The evaluation of the records was effected after data collection and comparative analysis with a standard amino acid chromatogram (SequencePro Applied Biosystems). The determined FVII sequence was compared to the amino acid theoretical sequence.

2 main sequences were systematically identified:
N terminal LC sequence: ANAFLEELRPGSLERECKEEQCSF (SEQ ID NO 3)
N terminal HC sequence: IVGGKVCPKGECPWQVLLLVNGAQLCG (SEQ ID NO 4
   3 other sequences, depending on the products, were identified:
LC sequence: LFWISYSDGDQ (SEQ ID NO 5) (atypical cleavage after lysine 38).
HC sequence: GATALELMVLNVPRLMTQ (SEQ ID NO 6) (atypical cleavage after arginine 290).
HC sequence: KVGDSP/VITEYMFCAGYSDGS (SEQ ID NO 7) (atypical cleavage after arginine 315).

The amino acids in bold and italics represent sequence gaps, that is to say amino acids that were not identified with the Edman sequencing because of the occurrence of posttranslational modifications such as γ-carboxylations, N- or O-glycosylations. A quantitative evaluation was effected so as to evaluate the amount of the various atypical cleavages depending on the FVII origin. The results are given in Table 1 hereunder:

TABLE 1
Various atypical cleavages in percent as compared to the
totality of the product, depending on the FVII origin.

| | FVII-pd | FVII-Tg | FVII-Tg | FVII-rec |
|---|---|---|---|---|
| Sequence | (%) | batch A (%) | batch B (%) | (%) |
| K₃₁₆VGDSP . . . | 27 | 17 | 52 | 9 |
| G₂₉₁ATALEL . . . | 8.5 | 8 | 13 | 4 |
| L₃₉FWISYS . . . | 12 | 26 | 8 | 4.5 |

FVII-pd: plasma-derived human FVII; FVII-Tg: non-mutated transgenic human FVII; FVII-rec: commercial recombinant human FVII (non mutated)

The FVII light chain has an atypical cleavage rate between the amino acids K38 and L39 varying from 4.5 to 26% depending on the product origin. The FVII heavy chain does possess an atypical cleavage rate between R315 and K316 (varying from 9 and 52% depending on the product origin) and is also cleaved between R290 and G291(varying from 4 to 13% depending on the product origin).

1-26. (canceled)

27. A human factor VII/VIIa modified as compared to the peptide sequence of the native human factor VII/VIIa, having at least two amino acids selected from lysine 38, arginine 290 and arginine 315 which are substituted or deleted, wherein:
said lysine 38 is replaced by an amino acid selected from the group consisting of glutamine, alanine, glutamic acid, glycine, isoleucine, leucine, methionine, histidine, phenylalanine, proline, serine, threonine, tryptophan, tyrosine and valine;
said arginine 290 is replaced by an amino acid selected from the group consisting of glutamine, alanine, glutamic acid, asparagine, glycine, isoleucine, leucine, methionine, histidine, phenylalanine, proline, serine, threonine, tryptophan, tyrosine and valine; and/or
said arginine 315 is replaced by an amino acid selected from the group consisting of glutamine, alanine, glutamic acid, asparagine, glycine, isoleucine, leucine, methionine, histidine, phenylalanine, proline, serine, threonine, tryptophan, tyrosine and valine.

28. The modified human factor VII/VIIa according to claim 27, wherein lysine 38 is replaced by an amino acid selected from the group consisting of glutamine, histidine and glutamic acid.

29. The modified human factor VII/VIIa according to claim 27, wherein asparagine 290 is replaced by an amino acid selected from the group consisting of glutamine, histidine, asparagine and glutamic acid.

30. The modified human factor VII/VIIa according to claim 27, wherein asparagine 315 is replaced by an amino acid selected from the group consisting of glutamine, histidine, asparagine and glutamic acid.

31. The modified human factor VII/VIIa according to claim 27, wherein lysine 38 is replaced by glutamine.

32. The modified human factor VII/VIIa according to claim 27, wherein arginine 290 is replaced by glutamine.

33. The modified human factor VII/VIIa according to claim 27, wherein arginine 315 is replaced by glutamine.

34. A nucleic acid encoding a modified human factor VII/VIIa as defined according claim 27.

35. An expression vector wherein is inserted a nucleic acid according to claim 34.

36. A cell transformed with a nucleic acid according to claim 34 expressing a modified human factor VII/VIIa.

37. A genetically modified organism comprising in its genome a nucleic acid encoding a modified human factor VII/VIIa as defined according to claim 27, and expressing said modified human factor VII/VIIa.

38. The genetically modified organism according to claim 37, which is a microorganism, an animal or a plant.

39. The genetically modified organism according to claim 37, which is a mammal.

40. The genetically modified organism according to claim 39, wherein said mammal is a female rabbit.

41. The genetically modified organism according to claim 37 which is an insect.

42. A method for preparing a human factor VII/VIIa comprising following steps of:
a) transforming a cell with a nucleic acid encoding a human
FVII/VIIa modified according to claim 27;
b) culturing the cell obtained in step a) so that said cell does express said factor VII/VIIa, and
c) purifying the modified human factor VII/VIIa expressed by the transformed cell cultured in step b).

43. A method for preparing a modified human factor VII/VIIa in the milk of a transgenic mammal, comprising following steps of:
a) providing a transgenic mammal which does express in its mammary glands a nucleic acid encoding a modified human factor VII/VIIa according to claim 27,
b) collecting the milk of the transgenic animal which contains factor VII/VIIa,
c) purifying the modified human factor VII/VIIa from said collected milk.

44. The method according to claim 43, wherein the transgenic animal is selected from the group consisting of a mouse, a female rat, a goat and a female rabbit.

45. The method according to claim 44, wherein the transgenic animal is a female rabbit.

46. A factor VII/VIIa composition, comprising a modified human factor VII/VIIa as defined according to claim 27.

47. A pharmaceutical composition comprising a modified human factor VII/VIIa as defined according to claim 27, and an excipient and/or a pharmaceutically acceptable carrier.

48. A method of preparing a drug comprising combining a factor VII/VIIa according to claim 27 with a pharmaceutically acceptable carrier.

49. A method treating clotting disorders, comprising administering an effective amount of a factor VII/VIIa according to claim 27 to a subject in need thereof.

50. A method for treating multiple hemorrhagic trauma, comprising administering an effective amount of a factor VII/VIIa according to claim 27 to a subject in need thereof.

51. A method for treating haemophilia, comprising administering an effective amount of a factor VII/VIIa according to claim 27 to a subject in need thereof.

52. A method for treating bleedings caused by anticoagulant overdosage, comprising administering an effective amount of a factor VII/VIIa according to claim 27 to a subject in need thereof.
